(19) United States
(12) Plant Patent Application Publication
(10) Pub. No.: US 2016/0037701 P1
(43) Pub. Date: Feb. 4, 2016

(54) Lavandula x intermedia 'Bridget Chloe'

(50) ;

(71) Applicant: John Thomas Hendon, JR., Blairsville, GA (US)

(72) Inventor: John Thomas Hendon, JR., Blairsville, GA (US)

(21) Appl. No.: 14/121,105

(22) Filed: Aug. 1, 2014

(51) Int. Cl. A01H 5/00

(52) U.S. Cl. PLT/445

(57) ABSTRACT

The genus of the claimed plant is Lavandula. The species it belongs to is ×intermedia, and the variety name is ‘Bridget Chloe’.

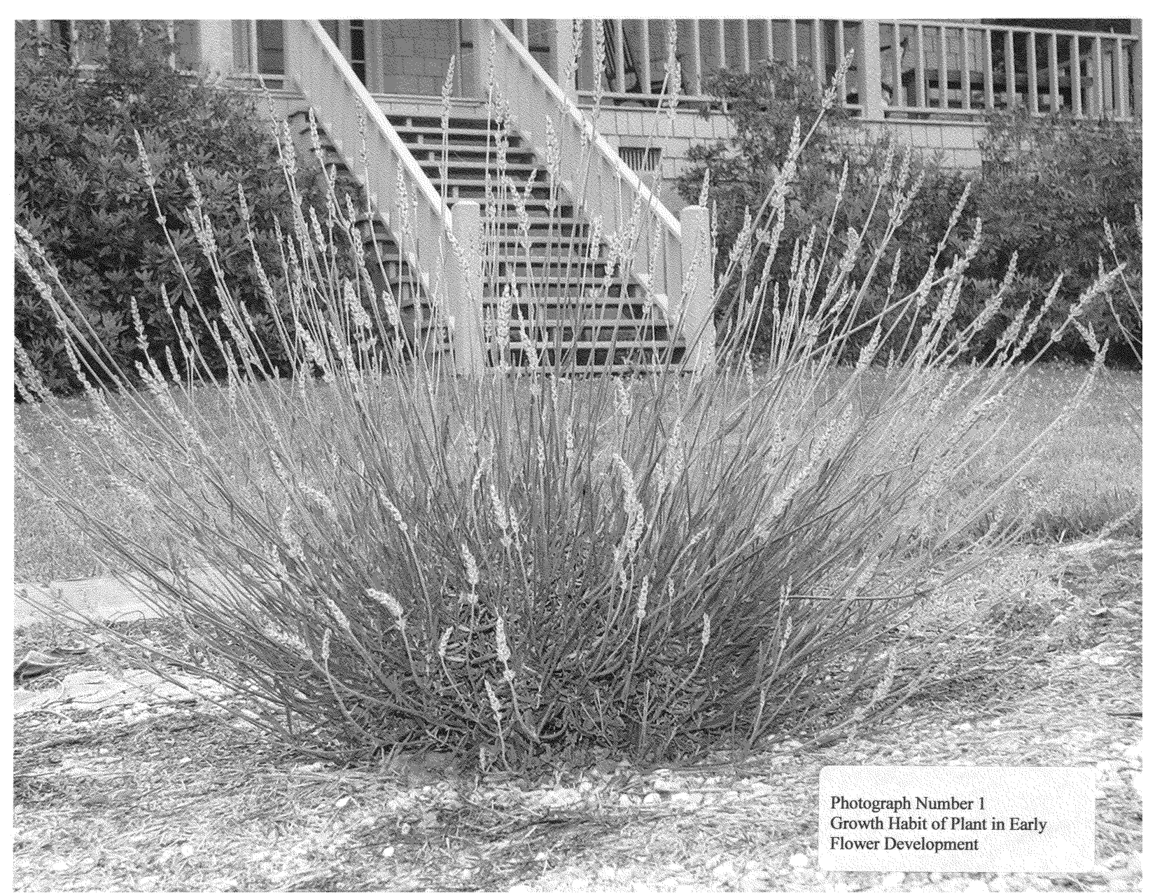

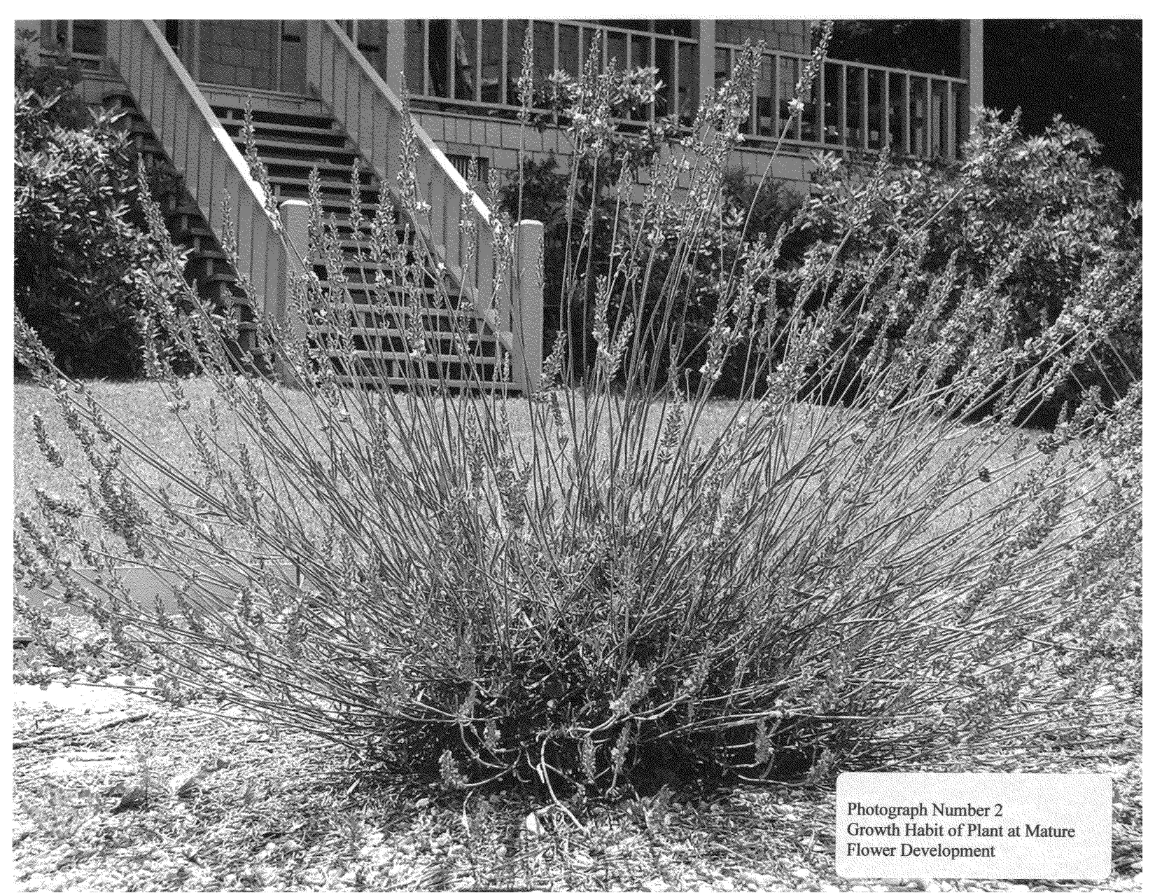

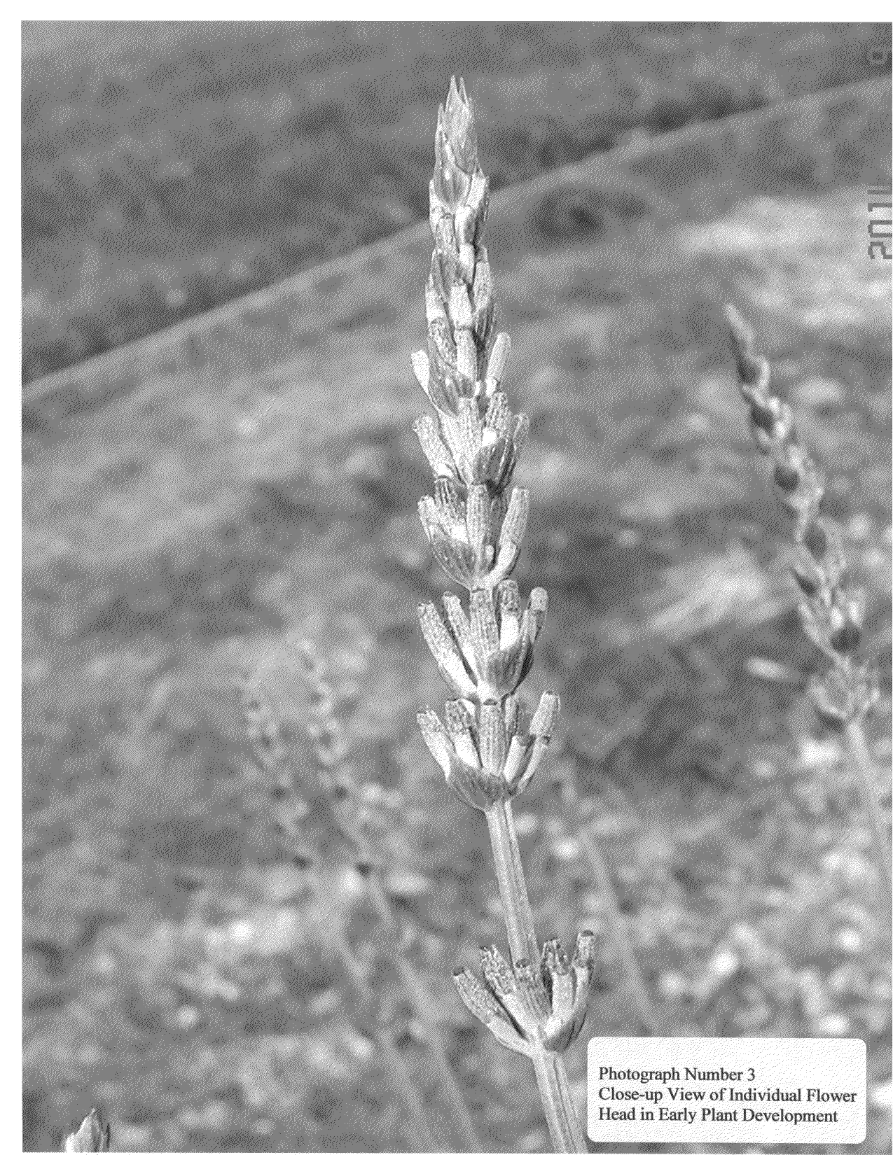

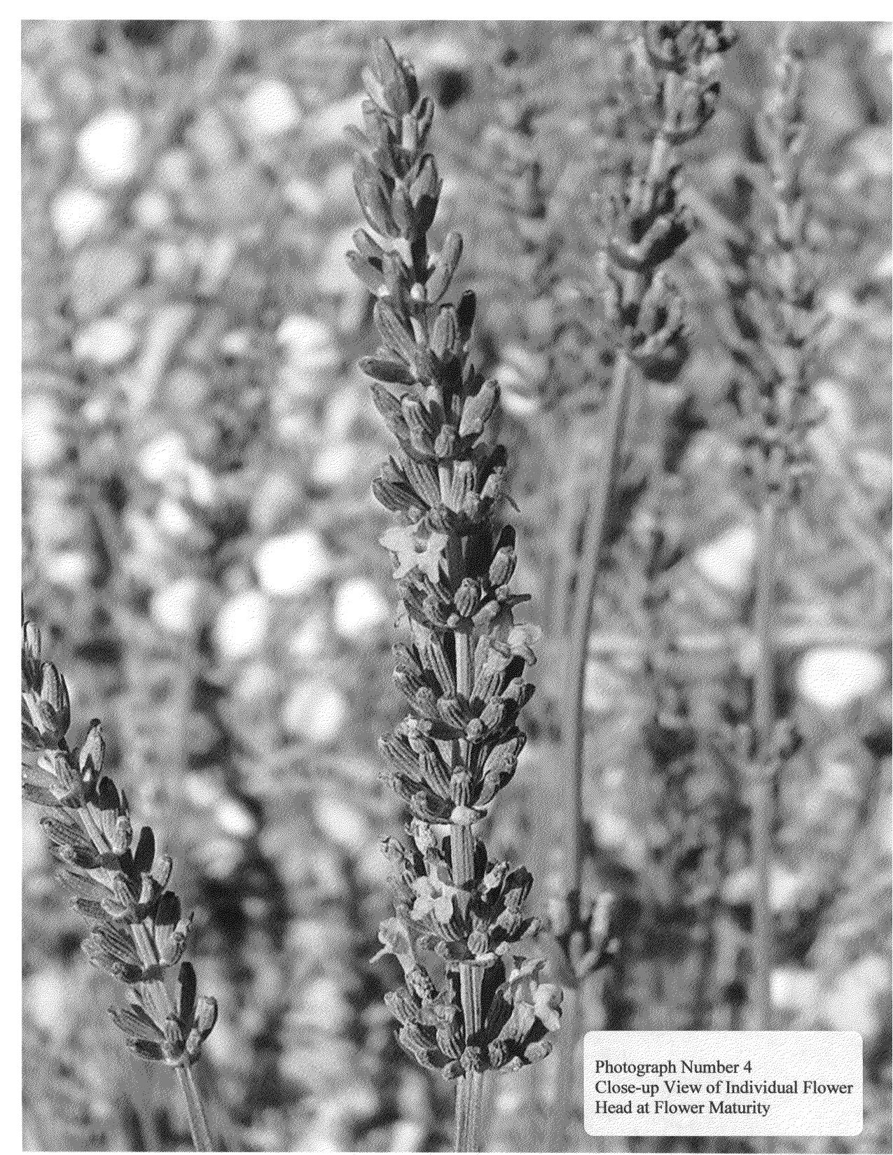

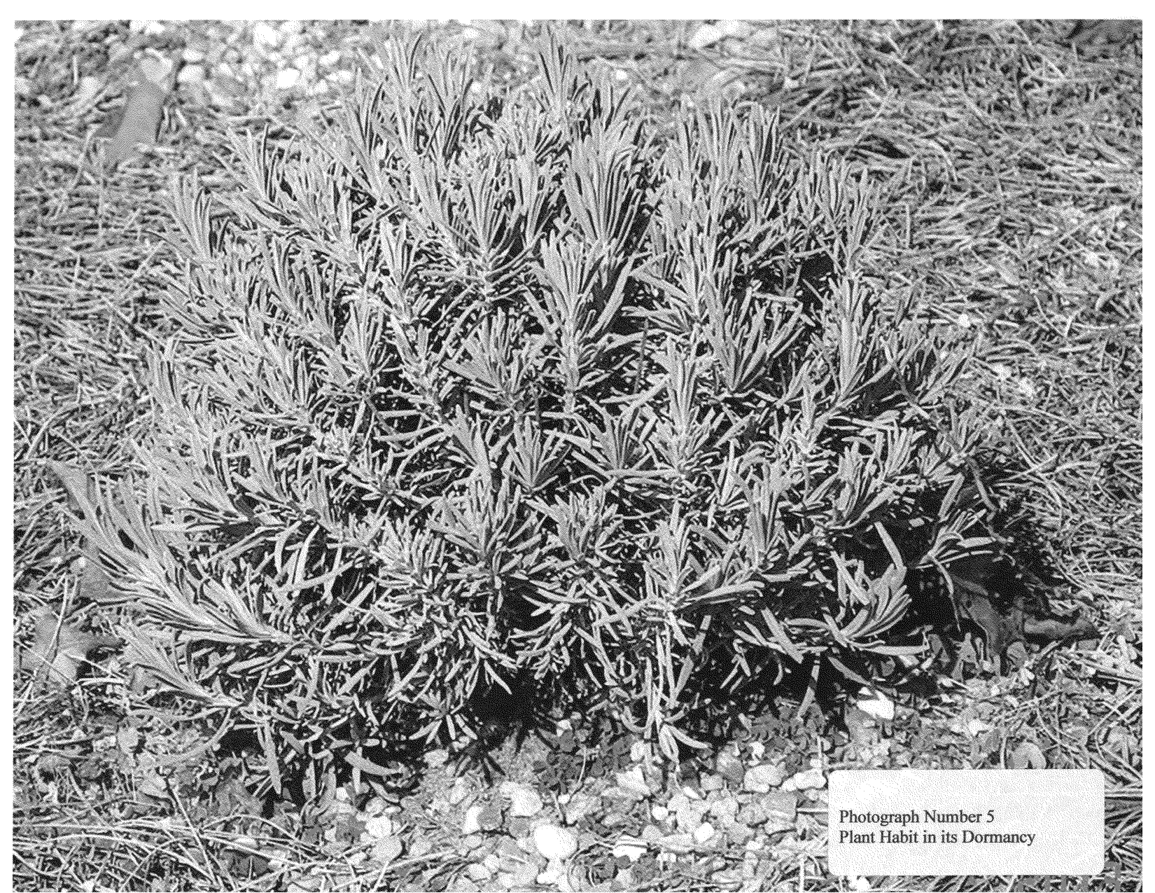

The parent plants from which this plant cultivar has been cloned were Lavandula×intermedia ‘Provence’.

The striking difference with this new plant variety (cultivar) is that it is very hardy in a hot, humid environment, with noticeably less foliage dieback in the summer months, and has low root rot occurrences. It has also demonstrated a strong resistance to both Lavender Leaf Spot and the Alfalfa Mosaic Virus.

The plant has a multi-faceted fragrance that begins firstly during flower spike development, with a hint or slight note (aroma) similar to that of black Cavendish tobacco, and then transforms through bud development to secondly, a desirable mild lilac note, and, lastly, a strong, rich, floral fragrance with a pleasing, non pungent, floral covered camphorous note. The rich floral fragrance continues to be observed in the dried flower buds from this plant, and it is long lasting. This multi noted aroma falls within the Aromatic Fougere fragrance category.

Immature flowers in this new plant variety (cultivar) display deep green⁵ flower bracts and green⁴ turning to dark purple³ calyxes whereas, mature flowers have desirable deep green⁵ bracks, dark purple³ calyxes, and deep purple¹ to vivid purple² corollas, unlike Provence and other Lavandin expressions in this market class.

The flower cluster (whorl) number per spike for the plant on average is high at 11.5, with up to fourteen clusters per spike not uncommon; and the flower bracts have an impressive 5 to 9 flowers per bract.

When harvested at peak calyx development, the dried flowers of the plant partially hold their desirable dark purple³ color, unlike the majority of the Lavendins which turn grayish in color.

### CROSS REFERENCE TO RELATED APPLICATIONS

There are no utility, co-pending or other original applications involved with this plant patent application.

### STATEMENT REGARDING FEDERALLY-SPONSERED RESEARCH AND DEVELOPMENT (IF ANY)

No Federally-sponsored research or development involvement was or is involved.

### LATIN NAME OF THE GENUS AND SPECIES OF THE PLANT CLAIMED

Genus: Lavandula

Species: ×intermedia

### VARIETY DENOMINATION

Cultivar variety: a new Lavandin, ‘Bridget Chloe’

### BACKGROUND OF THE INVENTION

1. Field of the Invention

The botanical name and market class of the invention is Lavandula×intermedia, which is often referred to as a Lavandin. A Lavandin occurs as a result of a cross between two plants in the lavender family, namely Lavandula angustifolia and Lavandula latifolia. The parent plants of the invention were the Lavandin, Provence. The intermediate features of the invention closely mimic that of the Lavandins which are known for their many morphological expressions. The discovery of this plant variety (cultivar), with its tolerance to heat, cold temperatures, and high humidity of the Georgia mountain region of the southeastern United States, would be offered to the nursery industry as a candidate for a lavender cultivar with high visual and complex fragrance attributes, and having a quality of high survival success in a temperate environment where sultry summer heat, moderately cold winters, and high humidity conditions exist.

2. Description of relevant prior art including information disclosed under 37 CFR 1.97 and 1.99

In the spring of 1998, six lavender plants (two Spanish, Lavandula stoeches; two Sweet Lavender, Lavandula angustifolia; and two Provence, Lavandula×intermedia) were planted in full to partial sunlight on a west facing slope on the applicant's property. In the winter of 1998/1999, dieback occurred in the two Sweet Lavender plants (approximately 20% in one and 40% in the other). One of the Spanish Lavender plants also had about 40% dieback. The two Provence Lavender plants did much better with only minor dieback splotches. In the following years, 2000 and 2001, the Sweet Lavender and the Spanish Lavender all but died out; and, therefore, these plants were pulled out and destroyed. The remaining two Provence Lavender plants, while somewhat shabby in appearance due to some dieback and woody growth occurring in the years from 1998 through 2001, were the original host plants from which cuttings were taken in the spring of 2002.

Plants from the cuttings, asexual reproductions from the two parent Provence plants numbering approximately thirty in total, were planted in late summer of 2002 in a prepared contour row located on a knoll, six to eight feet out and parallel to a woodland consisting of mixed hardwoods and pines. The woodland is located northeast of this cultivated contour row. Located to the southwest of this row (which runs from south to north) is an open meadow. The lavender in this row receives mid to late afternoon and evening sun from a western sun exposure. The elevation of the lavender row is 2,130 feet above sea level. In the early summer of 2003, following the initial plantings in 2002, subsequent cuttings were taken from the earlier cloned plants in this row of lavender and placed back into the row in the fall of 2003 in order to fill the row out.

In June of 2010, it was noticed that some of the plants in this now somewhat older row of lavender plants showed a deeper purple¹* color in their flower heads than the majority of the remaining plants. Upon investigating these plants, it was also observed that they had a more deeply floral fragrance than the remaining plants. Because they show a significant characteristic difference from the original parent plants and the other plants in the row that were from the same parent plants, a lavender variety (cultivar) study was conducted over a four year period. No association with the original Spanish or Sweet Lavender plants is apparent in the plantings, and only the two original Provence Lavender plants were used in the initial cuttings. * Note: color references, numbering 1 through 12, are found under “Color Key Reference”.

Results from the study conducted by the applicant, along with the presence of the actual progression of cloned plantings (asexual reproductions) on the above property, support a new lavender variety (cultivar) presence. All subsequent plants from cuttings of preceding plants have maintained the same differing and desirable characteristics from that of the original parent Provence Lavender plants. It is believed that the new plants discovered in 2010 and the subsequent re-propagated offspring plants from asexual cuttings are a form of a mutation occurrence which resulted in a new variety (cultivar) of Lavandula×intermedia that occurred at some point in time between the date of the first plantings in the year 2002 and the year 2010, the year the cultivar was first discovered. This occurrence, resulting from natural and/or environmental conditions in the field, therefore, produced new morphological expressions in the clones taken from the initial Provence Lavender plants.

### SUMMARY OF THE INVENTION

While this new lavender cultivar shares some attributes with other lavender varieties, such as Lavandula×intermedia ‘Dutch’; Lavandula×intermedia ‘Abrialii’; and Lavandula×intermedia ‘Provence’, from which the subject lavender plants were propagated; the overall characteristics of this new plant are notably different from the aforementioned varieties (cultivars). Also, it should not be confused with Gros Blue, a relatively new Lavandin cultivar from France, with near navy blue flower heads, in lue of deep purple¹ to vivid purple² flower heads. Nor should it be confused with Lavandula×intermedia ‘Phenomenal’, introduced in 2012 by Peace Tree Farm, Kintnersville, Pa., which has similar attributes, but with bluer bud color and light green to silver-gray leaves.

The flower spikes in this new plant variety (cultivar) are not quite as narrow as Dutch Lavender, and the flower heads are a deep purple¹ to vivid purple² with strikingly purple³ calyxes in early flower development, unlike either Dutch Lavender or Provence Lavender which have violet-green calyxes. Also, the leaves are not as silvery-green and broad as Dutch Lavender. Furthermore, this new cultivar has proven to be hardy and disease free throughout the test period, whereas Abrialii, a close look alike, is subject to disease, has short plant life, has not been planted or introduced in anyway to the property proper; and has purple to gray-blue calyxes instead of the green⁴ turning to dark purple³ calyxes of the new ‘Bridget Chloe’ cultivar. In addition, the subject lavender's dried flower buds tend to be more elongated and just a bit thinner, are more richly floral in fragrance, and are not as easily separated from the flower spikes as is Provence Lavender. The dried flower buds from Provence Lavender for the most part are plump, very fragrant with a camphorous overtone, easily separate from the flower spikes, and turn gray as in the case for the majority of the Lavandins. On the other hand, when harvested at peak calyx development, this new plant cultivar's dried flower buds partially hold their dark purple³ color as in the case for Lavandula×intermedia ‘Grosso’ which dries to a similar dark purple color.

The difference with the new plant variety (cultivar) of Lavandula×intermedia ‘Bridget Chloe’ is, that when the flower spikes are developing, its fragrance begins firstly, with a hint or slight note (aroma) similar to that of black Cavendish tobacco, and then transforms through bud development to secondly, a desirable mild lilac note, and, lastly, a strong, rich, floral fragrance with a pleasing, non pungent, floral covered camphorous note. Immature flowers in the new plant variety (cultivar) display deep green⁵ flower bracts and green⁴ turning to dark purple³ calyxes; whereas, mature flowers have desirable deep green⁵ bracts, dark purple³ calyxes, and deep purple¹ to vivid purple² corollas. However, it should be noted that if this plant receives less than full sun exposure (is partially shaded); the developed flower calyxes turn from dark purple³ back to green⁴, less the fringes, upon development of vivid purple² corollas. Thus, the more sun exposure, the darker the flower color in the cultivar. Provence lavender buds are more uniformly purple⁶ throughout development, with from green⁴ to brown⁷ bracts at full maturity.

Furthermore, this new plant variety (cultivar) makes for an impressive ornamental and/or row crop with an easily maintained mounded habit and has proven to be extremely hardy in the field as propagated and grown in Union County, Ga., rendering it suitable for planting in high humidity areas of the southeastern United States, USDA Zone-7; and, a strong likelihood of being suitable for Hardiness Zones 5 through 9 as is the case for the parent plants.

### BRIEF DESCRIPTION OF THE DRAWING (PHOTOGRAPH)

Number 1: Growth Habit of Plant in Early Flower Development

Depicts growth habit of the new plant cultivar in the early month of June, when the flower spikes are still developing. Note: uniform, compact habit of overall plant; and deep green^{5**} foliage. ** A deeper green than that found in Provence Lavender.

Number 2: Growth Habit of Plant at Mature Flower Development

Depicts growth habit of the new plant in mid to late June, when flower spikes are fully developed. Note: plants mature flower heads; the spraying outwards, erect/semi-aslope growth form; and continued uniform, compact habit of the plant.

Number 3: Close-up View of Individual Flower Head in Early Plant Development

Depicts the individual parts of the plant's flowers in early plant development. Note: deep green⁵ flower bracts; and gree⁴ turning to dark purple³ calyxes.

Number 4: Close-up View of Individual Flower Head at Flower Maturity

Depicts the individual parts of the plant's flowers at flower maturity. Note: dark purple³ calyxes; and deep purple¹ to vivid purple² corollas.

Number 5: Plant Habit in its Dormancy

Depicts plant's habit in its dormancy. Note: bluish¹⁰, silver-green⁸ to grayish¹¹ color of the foliage; and the compact habit.

### DETAILED DESCRIPTION OF THE PLANT

The subject new plant variety (cultivar) belongs to the Lamiaceae (mint) family. The genus is Lavandula. The species it belongs to is ×intermedia, and the variety name is ‘Bridget Chloe’. The full name for this plant is Lavandula×intermedia ‘Bridget Chloe’. The species or market class for this plant is also referred to as a Lavandin. The parent plants from which this Plant cultivar has been cloned were Lavandula×intermedia ‘Provence’. This plant is a clone, grown from cuttings rather than from seeds. Characteristic of the Lavendins, it has a highly floral fragrance.

At its maturity the subject plant grows to a height of from 61.6 to 76.8 centimeters (2.0 to 2.5 feet), and has a width of from 76.8 to 107.8 centimeters (2.5 to 3.5 feet). Unlike Provence Lavender, the foliage has a deeper green⁵ color in the spring and early summer months. In mid to late summer, the foliage exhibits a silver-green⁸/gray⁹ color. Provence Lavender has a lighter green⁴ hue to its color at this time of year. In the fall and winter the new cultivar has distinct bluish¹⁰, silver-green⁸ to grayish¹¹ foliage; whereas, Provence Lavender has a grayish¹¹-green⁴ color in these latter seasons. The bluish¹⁰ color of the new cultivar is more noticeable under shade or on overcast days. The individual, long, silver-green⁸ to gray⁹, narrow, lanceolate leaves of the new cultivar during the dormancy period closely resemble the color and structure of the leaves of Provence Lavender, and with the naked human eye, are not readably distinguishable from that of Provence Lavender.

The habit of the invention is a mounded shrub, and has a more even spread than Provence lavender. It has long flowering stems with attractive conical flower spikes consisting of deep purple¹ to vivid purple² flowers, rather than the uniform purple⁶ flowers found in the Provence lavender spikes. There are, in the case of Lavandins, occasional axial flower shoots occurring on the flower stems of the new plant cultivar. Also, of note, the new plant cultivar has deep green⁵ bracts throughout the flower bloom, even when the corollas are turning brown¹². Provence Lavender, on the other hand, has green⁴ turning to brown⁷ bracts during flower development.

The striking difference with this new plant variety (cultivar) is that it is very hardy in a hot, humid environment, with noticeably less foliage dieback in the summer months, and has low root rot occurrences. Also, this new cultivar has demonstrated a strong resistance to both Lavender Leaf Spot and the Alfalfa Mosaic Virus.

Furthermore, what sets this new plant cultivar of Lavandula×intermedia apart, is that when the flower spikes are developing, its multi-faceted fragrance begins firstly, with a hint or slight note (aroma) similar to that of black Cavendish tobacco, and then transforms through bud development to secondly, a desirable mild lilac note, and, lastly, a strong, rich, floral fragrance with a pleasing, non pungent, floral covered camphorous note. This strong, rich, floral fragrance continues to be observed in the dried flower buds from this plant, and it is long lasting. This multi noted aroma falls within the Aromatic Fougere fragrance category.

Most notably, immature flowers in this new plant variety (cultivar) display the deep green⁵ flower bracts and green⁴ turning to dark purple³ calyxes whereas, mature flowers have desirable deep green⁵ bracks, dark purple³ calyxes, and deep purple¹ to vivid purple² corollas, unlike Provence and other Lavandin expressions in this market class. When this plant receives less than full sun exposure (is partially shaded); the developed flower calyxes turn from dark purple³ back to green⁴, less the fringes, upon development of vivid purple² corollas. Thus, the more sun exposure, the darker the flower color in the cultivar. Provence lavender buds are more uniformly purple⁶ throughout development, with from green⁴ to brown⁷ bracts at full maturity.

For this plant, the average length of the flowering stems is from 35.9 to 44.4 centimeters (14.0 to 17.3 inches) above foliage. The plant has a spraying outwards, erect/semi-aslope, growth form. It has an average flowering terminal spike length of 8.5 centimeters (3.3 inches) without the lower cluster included, and 11.6 centimeters (4.5 inches) with the lower cluster included; an average flower cluster (whorl) number per spike of 11.5, with up to fourteen not uncommon; and a flower number of 5 to 9 per bract, with from 10 to 18 per cluster. The deep green⁵ leaf-like bracts are ovate-rhombic in form and have a width to length ratio of 2.00 to 3.20. Typically, the bracts are opposite paired, with alternate 90° placement along the length of the conical flower spike. The tubular dark purple³ calyx is strongly ribbed with thirteen veins and is five-toothed. The lower clusters are more separated with the lowest being from 2.6 to 9.0 centimeters (1.0 to 3.5 inches) below the upper clusters. The deep purple¹ to vivid purple² corolla is typically two-lipped.

Other attributes include the following: It is a perennial herb; is an impressive ornamental; is highly vigorous and produces numerous flower spikes; can be used for both cut and dried flowers; is suitable for border or group plantings and row crops; is culinary; and is both rabbit and deer resistant.

*Color Key Reference Using “NBS-ISCC Centroids by Number”

| Text Color Name & Ref. No. | NBS-ISCC No. | NBS-ISCC Color |
|---|---|---|
| “deeper or deep purple” ¹ | 219 | Deep Purple |
| “vivid purple” ² | 216 | Vivid Purple |
| “strikingly or dark purple” ³ | 220 | Very Deep Purple |
| “green” ⁴ | 117 | Strong Yellow Green |
| “deep or greener” ⁵ | 118 | Deep Yellow Green |
| “purple” ⁶ | 194 | Vivid Purplish Blue |
| “brown” ⁷ | 76 | Light Yellowish Brown |
| “silver-green” ⁸ | 154 | Light Greenish Gray |
| “gray” ⁹ | 190 | Light Bluish Gray |
| “bluish” ¹⁰ | 186 | Grayish Blue |
| “grayish” ¹¹ | 191 | Bluish Gray |
| “brown” ¹² | 57 | Light Brown |

1. This Claim is for a new lavender plant cultivar (Lavandin) named Lavandula×intermedia ‘Bridget Chloe’, which exhibits unique qualities of both different and beneficial attributes for the plant's market class, consisting of exciting new color exhibits, pleasing rich floral fragrance, and plant hardiness.
